(19) United States
(12) Patent Application Publication
(10) Pub. No.: US 2008/0221563 A1
(43) Pub. Date: Sep. 11, 2008

(54) Safety Device For a Hf-Surgery Appliance

(76) Inventor: Jurgen Beller, Gomaringen (DE)

Correspondence Address: DICKSTEIN SHAPIRO LLP, 1825 EYE STREET NW, Washington, DC, 20006-5403, US

(21) Appl. No.: 12/064,802

(22) PCT Filed: Aug. 8, 2006
(86) PCT No.: PCT/EP2006/007844

§ 371 Date: Feb. 25, 2008

(30) Foreign Application Priority Data
Aug. 26, 2005 (DE) 10 2005 040 487.1

(51) Int. Cl. A61B 18/12

(52) U.S. Cl. 606/34

(57) ABSTRACT

A safety device for a high-frequency surgery appliance that includes at least one high-frequency generator with an active output and a neutral output to generate a working current, and at least two working connectors. At least one instrument, through which the working current is conducted to a biological tissue, is connected to respective working connectors. The safety device reduces the danger presented by fault currents that may flow through an inactive instrument. The safety device is connected to the active output as well as to the neutral output in such a way that every working connector through which no working current is flowing is connected only to the neutral output.

### FIELD OF THE INVENTION

Disclosed embodiments relate to a safety device for a high-frequency (HF)-surgery appliance, and in particular to a safety device that reduces the danger presented by fault currents that can flow through an inactive instrument.

### BACKGROUND OF THE INVENTION

In electrosurgical appliances that operate at high frequency (HF surgery appliances) alternating currents in the range of 300 kHz are employed. In this frequency range capacitive and inductive couplings play a considerable role.

When several instruments are attached to a HF-surgery appliance, for example one for cutting and another for coagulation, the capacitive couplings can allow currents to flow through the “inactive” leads. If these leads were to make contact with the patient or the surgeon, injuries could result.

The IEC standard 60601-1-6 describes measurement procedures with which to determine the limiting value of such a coupled current with respect to the earth potential or to the opposite electrode. This limiting value is 150 mA.

The document DE 35 23 871 C3 discloses a safety circuit for a HF-surgery instrument that is intended to provide a means by which, if leakage currents should appear, they can be prevented as far as possible from putting the patient at risk of injury. This known device is relatively elaborate.

Thus, a simple safety device that allows leakage currents to be avoided is desired.

### SUMMARY

The objective of the present invention is to disclose a safety device for a HF-surgery appliance that, by simple means, allows leakage currents to be avoided as completely as possible.

In particular, the present safety device for a HF-surgery appliance that comprises at least one HF generator with an active output and a neutral output to generate a working current, and at least two working connectors to each of which can be connected, by way of electrical leads, at least one instrument through which the working current can be conducted to a biological tissue, such that the safety device at least reduces the danger presented by unintended “fault currents” that can flow through an inactive instrument. The safety device is connected to the working connectors as well as the neutral output in such a way that every working connector through which no working current is flowing is connected to the neutral output.

Hence, in the disclosed embodiments, fault currents or leakage currents are not restricted but rather are “annihilated.” Thus the risk that injury will be caused by the appliances is reduced.

The safety device preferably comprises switching elements with which to switch a working connector from the neutral output to an active output of the HF generator and to conduct the working current from the HF generator to an instrument. Such changeover switches are preferably constructed as relays and hence are simple to manufacture and to operate.

A control means is provided which is preferably constructed as a microcomputer designed so that in response to an activation signal, the working connector of an instrument to be activated is first connected to an active output of the HF generator. Then, the HF generator is triggered so that it delivers the working current. Hence, at the outset, none of the working connectors are connected to the HF generator; instead, all of them are at the neutral potential. Furthermore, when switching is initiated, the working current need not immediately flow through the switch, which instead changes state while in a no-current condition. Only after switching has occurred is the HF generator triggered so as to deliver a “signal” set by the user, i.e. the actual working current.

There are preferably provided sampling elements at the working connectors to monitor the current and/or voltage. The sampling elements are designed to generate a fault-current signal when the current and/or the voltage at a working connector through which no working current is flowing exceeds a pre-adjustable threshold. In this way, it is easy to determine whether any fault currents are present. If that is the case, then appropriate measures can be taken. In particular, in such a case where the threshold is exceeded, a warning signal can be emitted by a signal element to announce an (excessive) fault current. This makes it possible for the user easily to check the operational state of the appliance. When this threshold is reached, or in certain cases a second, higher threshold, the HF generator can be switched off by an appropriate control signal, as a result of which the operational safety of the appliance is further increased.

### BRIEF DESCRIPTION OF THE DRAWINGS

In the following, the invention is described with reference to an exemplary embodiment, which is explained in greater detail with reference to the enclosed drawing.

FIG. 1 illustrates a high-frequency surgery appliance incorporating the safety device of the disclosed embodiments.

### DETAILED DESCRIPTION OF THE INVENTION

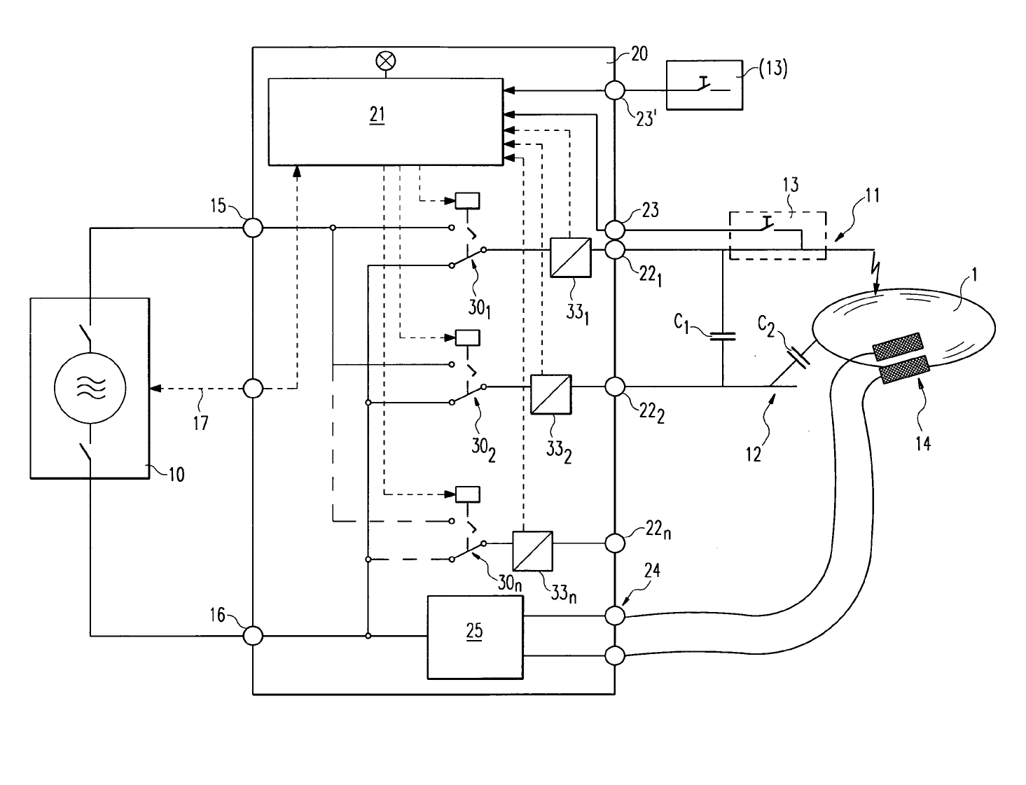

Referring to FIG. 1, a high-frequency surgery appliance incorporating the safety device 20 of the disclosed embodiments is shown. A high-frequency (HF) generator 10 is provided, which can be adjusted by way of the customary adjustment organs in a manner known in the art. The HF generator 10 includes an active output 15 and a neutral output 16. The neutral output 16 is connected to an indifferent electrode 14 by way of a test-circuitry component 25 and a connector 24. The indifferent electrode 14 is attached to the tissue 1 of a patient in such a way as to ensure that only a slight density of current will be transferred from the indifferent electrode 14 into the tissue 1. The safety of the contact is tested by the test circuitry 25.

The signal from the active output 15 is sent to one pole of each of the change-over switches 30₁ to 30ₙ. The second pole of each switch 30₁ to 30ₙ is connected to the neutral output 16.

The third pole (input) of the change-over switches 30₁ to 30ₙ is connected to working connectors 22₁ to 22ₙ of the safety device 20 by way of transformers 33₁ to 33ₙ. The transformers 33₁ to 33ₙ ascertain which currents are flowing through the working connectors 22₁ to 22ₙ, or which voltages are present at them.

In the exemplary embodiment an active instrument 11, i.e. one that is currently being used, is attached to a first working connector 22₁. In order to allow a high-frequency current to flow, e.g. for the purpose of cutting, the user actuates a switch 13 either located on the active instrument 11 or being a pedal switch (13), which is connected by way of a control connector 23 or 23′, respectively, to a control means 21 that is preferably designed as a microprocessor. The control means 21 receives the signals from the switch 13 and the transformers 33₁ to 33ₙ. In addition, by way of a control lead 17, the control means 21 is connected to the HF generator 10 in such a way that the HF generator 10 is controlled by the control means 21 and the control means 21 is informed about operational states of the HF generator 10. Furthermore, the control means 21 is in controlling communication with the change over switches 30₁ to 30ₙ, so that in response to relevant signals from the control means 21, the working connectors 22₁ to 22ₙ can be switched by the change-over switches 30₁ to 30ₙ either to the active output 15 or to the neutral output 16. The change over switches 31₁ to 30ₙ may be constructed, for example, as relays.

During employment of the appliance, when the switch 13 is actuated, the control means 21 changes the state of the change-over switch 30₁ in such a way that the working connector 22₁ is applied to the active output 15 of the HF generator 10. The other change-over switches 30₂ to 30ₙ remain in the position shown in the drawing, so that the working connectors 22₂ to 22ₙ remain in communication with the neutral output 16. After a pre specified time has elapsed, the control means 21 controls the HF generator 10 so that the HF generator 10 supplies a working current to the active instrument 11 by way of the change-over switch 30₁, the transformer 33₁ and the working connector 22₁. The pre specified time is one that is as short as possible while still being long enough to ensure reliable switching of the change-over switches 30₁ to 30ₙ. The current circuit is closed by the tissue 1, the indifferent electrode 14 and the test circuitry 25, terminating at the neutral output 16 of the HF generator 10.

When the current flows in this way, currents can be coupled into an inactive instrument 12, or its connector fork, by way of capacitive couplings C₁ and C₂, shown schematically in the drawing. When such a current is coupled into the inactive instrument 12, it flows through the working connector 22₂, the transformer 33₂ and the change over switch 30₂ to the neutral output 16 of the HF generator 10 or to the indifferent electrode 14. This arrangement prevents any damaging current from flowing through the inactive instrument 12, even if it comes into contact with the user or the patient.

If the fault current (measured by the transformer 33₂) exceeds a threshold that has been preset in the control means 21, the latter emits an alarm signal (shown here as a warning lamp). Alternatively or in addition (if a still higher threshold is exceeded) the control means 21 switches off the HF generator 10.

1. A safety device for a high-frequency surgery appliance that comprises at least one high-frequency generator with an active output and a neutral output to generate a working current, and at least two working connectors for connection of at least one instrument through which the working current can be conducted to a biological tissue, such that the safety device reduces the danger presented by fault currents that can flow through an inactive instrument, wherein the safety device is connected to the at least two working connectors and the neutral output such that each working connector through which no working current is flowing is connected to the neutral output.

2. The safety device according to claim 1, further comprising change-over switches for switching respective working connectors from the neutral output to the active output of the high-frequency generator and conducting a working current from the high-frequency generator to an instruments.

3. The safety device according to claim 1, further comprising control means configured such that in response to an activation signal, the working connector of an instrument that is to be activated is connected to the active output of the high-frequency generator, and then the high-frequency generator is controlled so that it supplies the working current.

4. The safety device according to claim 1, further comprising sampling elements provided at the working connectors, the sampling elements detecting the magnitude of a current and/or a voltage, and generating a fault-current signal whenever the current and/or the voltage at a working connector through which no working current is flowing exceeds a predetermined threshold.

5. The safety device according to claim 4, further comprising a signal element that emits a warning signal in response to the fault-current signal.

6. The safety device according to claim 4, wherein the sampling elements are in controlling communication with the high-frequency generator, so that in response to the fault-current signal the high-frequency generator is switched off.

7. A high-frequency surgery appliance comprising:
at least one high-frequency generator having an active output and a neutral output and configured to generate a working current;
a safety device connected to the active output and the neutral output;
at least one instrument through which the working current is conducted to a biological tissue; and
at least two working connectors connecting the safety device to the at least one instrument,
wherein the safety device is configured such that the neutral output is connected to each of the at least two working connectors through which no working current is flowing.

8. The high-frequency surgery appliance according to claim 7, wherein the safety device further comprises at least two change-over switches, corresponding respectively to the at least two working connectors, wherein the change-over switches are configured to switch the corresponding working connector from the neutral output to the active output and are further configured to conduct the working current from the high-frequency generator to the respective at least one instrument.

9. The high-frequency surgery appliance according to claim 7, wherein the safety device further comprises control means configured such that in response to an activation signal, the working connector corresponding to an instrument that is to be activated is connected to the active output of the high-frequency generator, and then the high-frequency generator supplies the working current.

10. The high-frequency surgery appliance according to claim 7, wherein the safety device further comprises at least two sampling elements provided at respective working connectors, wherein the sampling elements detect the magnitude of a current and/or a voltage and generate a fault-current signal when the current and/or the voltage at a particular working connector through which no working current is flowing exceeds a predetermined threshold.

11. The high-frequency surgery appliance according to claim 10, wherein the safety device further comprises a signal element that emits a warning signal in response to the fault-current signal.

12. The high-frequency surgery appliance according to claim 10, wherein the sampling elements are in controlling communication with the high-frequency generator, such that in response to the fault-current signal the high-frequency generator is switched off.

13. A safety device for use with a high-frequency surgery apparatus, the safety device comprising:
a connection to an active output of a high-frequency generator;
a connection to a neutral output of the high-frequency generator;
a plurality of working connectors for respectively connecting to at least one instrument through which a working current is conducted to a biological tissue;
a plurality of change-over switches corresponding respectively to the plurality of working connectors, wherein the change-over switches are configured to switch the corresponding working connector from connection to the neutral output to connection to the active output;
a control means configured to, in response to an activation signal, cause the change-over switch corresponding to a particular instrument that is to be activated to connect the working connector corresponding to the particular instrument to the connection to the active output and further configured to signal the high-frequency generator to supply the working current; and
a plurality of sampling elements corresponding respectively to the plurality of working connectors, wherein the sampling elements detect the magnitude of a current and/or a voltage at respective working connectors and generate a fault-current signal when the current and/or the voltage at a particular working connector through which no working current is flowing exceeds a predetermined threshold.

14. A method of reducing fault currents flowing through an inactive instrument in a high-frequency surgery apparatus comprising:
initializing a plurality of change-over switches in a safety device such that the plurality of change-over switches are connected to a neutral output of a high-frequency generator, thereby causing all of a plurality of instruments of the high-frequency surgery apparatus to be connected to the neutral output;
in response to an activation signal, switching the change-over switch corresponding to an instrument to be activated such that the instrument to be activated is connected to an active output of the high-frequency generator; and
supplying a working current to the instrument that is to be activated,
wherein any fault currents that may be produced at inactive instruments are caused to flow through the respective change-over switch and the neutral output of the high-frequency generator rather than into an external source.

15. The method of claim 14, wherein the safety device further comprises means for detecting the magnitude of a current and/or a voltage flowing to a working connector connecting a particular instrument to the safety device, and the method further comprises generating a fault-current signal when the current and/or the voltage at a particular working connector through which no working current is flowing exceeds a predetermined threshold.

16. The method of claim 15, further comprising causing a signal element to emit a warning signal in response to the fault-current signal.

17. The method of claim 15, further comprising switching off the high-frequency generator in response to the fault-current signal.
